(19) United States
(12) Patent Application Publication
(10) Pub. No.: US 2004/0248325 A1
(43) Pub. Date: Dec. 9, 2004

(54) Method for simultaneous multiple probes/multiple targets screening procedure

(76) Inventor: Cuneyt Bukusoglu, Norwood, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/484,220

(22) PCT Filed: Jul. 19, 2002
(86) PCT No.: PCT/US02/23137

Related U.S. Application Data
(60) Provisional application No. 60/307,062, filed on Jul. 19, 2001.

(51) Int. Cl. G01N 33/53

(52) U.S. Cl. 436/548

(57) ABSTRACT

Monoclonal antibodies that are specific for a physiological disorder of interest are used to conduct an immunometric assay for antigens correlated with that specific disorder. The invention provides a method which comprises a process having one probe and one target detection. Multiple probes are applied tissue samples without destroying the integrity of the tissue sample. The inventive method utilizes a specific reagent system that is a Probe-Tagging system. This system utilizes a cleavable linker and a specific tagging molecule that upon dissociation may be analyzed using micro-array-based detection systems or other detection methods known within the art.

RELATED APPLICATIONS

This application claims the priority filing benefit of U.S. provisional patent application 60/307,062, filed on Jul. 19, 2001, which is incorporated in its entirety by reference.

### FIELD OF INVENTION

The present invention relates generally to the detection of molecules, such as biological molecules. More specifically, the present invention relates to a method utilizing multiple tagged probes on human and animal tissue specimens. In one illustrative embodiment, probes having cleavable tags are used to identify multiple targets on tissue sections. The use of cleavable and non-cleavable probes allows establishment of target profiling for tissue samples.

### BACKGROUND OF INVENTION

The utilization of human tissue sections for diagnostic and prognostic applications has been common practice for the past 50 years. The main areas that utilize tissue sections for diagnostic and/or prognostic applications are Histochemistry, Immunohistochemistry (IHC), and in-situ Hybridization (ISH). In routine practice, tissue samples are obtained via biopsies (e.g. breast cancer screening) or during surgeries and autopsies. These tissue samples are kept frozen or processed in certain ways preventing post mortem changes (autolysis) and preserving all cell components (cell membrane, nucleus, mitochondria, etc). The processing of these tissue sections protects cell components by hardening soft tissue and converting the normal semi-fluid portion of the cells to an irreversible semi-solid consistency thereby allowing easy manipulation during subsequent processing.

One of the most common tissue processes is “Formalin Fixation” which is used to crosslink all available target sites followed by “Paraffin Embedding” providing a solid mass for easy sectioning, probing and manipulation. Unfortunately, in almost all routine clinical diagnostic laboratories each tissue section is used for the detection of only one target molecule. For example, a typical breast cancer antibody screening panel includes ER, PR, cerbB-2, Ki67, and p53. For each patient, five tissue sections (one for each antibody used) are needed to complete the panel. While it is possible to use two different probes, this is usually done in research applications where time limitation is not an issue. In “two-probe” applications, different probes (mouse/rabbit antibodies) and different labels (HRP/Alkaline Phoshatase) are usually utilized. The main difficulty with two-probe tests occurs during evaluation where chromogenic precipitation usually overlaps and thus limits the visualization of some target molecules.

Additionally, traditional screening methods are labor-intensive and are based upon subjective microscopic evaluation. In routine practice, each stained tissue section is screened by a histotechnologist and evaluated by a pathologist. This screening is done microscopically by observing staining profile and morphological topography, and comparing these features to known normal and abnormal tissue sections. Unfortunately, this labor intensive method is both costly and prone to differing interpretations. Furthermore, the overall process is extremely time consuming from tissue preparation to staining, counter staining, and coverslipping to evaluation.

Most importantly, the results of these traditional methods are subjective with differing results. The personal conclusions of pathologist will differ due to different schools of education, training and personal point of views. Additionally, each slide is evaluated within traditional methods according to one or two target molecules. There is limited quantitative or semi-quantitative point of reference using traditional methods. While current systems utilizing image-based analysis offer an improved, unbiased evaluation such as Chroma Vision Medical Systems Inc. (San Juan Capistrano, Calif.) automated cellular imaging system (ACIS), these methods have limited capacity with respect to the number of different target molecules they can simultaneously analyze (i.e. one or two probes).

While traditional methods are still widely used, current technologies have been developed to address problems existing within traditional methods. One solution has been microarray based hybridization and detection system by Affymetrix Inc., as described within U.S. Pat. Nos. 5,445,934, 5,744,305, 6,261,776, 6,291,183, 5,700,637, 5,945,334 and European Patent EP 619,321,203. These patents have resulted in the GeneChip® Expression Analysis system which consists of high-density oligonucleotide containing microarrays, “probe arrays” and biotin-labeled mRNA or cDNA “target”. In this system biotin-labeled cDNA is prepared from mRNA extracted from target tissues and hybridized with a probe array. DNA chips are small flat surfaces on which strands of one-half of the DNA double-helix-called DNA probes or oligos are bound. Since one-half of the DNA double-helix naturally binds with its complementary other half, a process called hybridization, this type of chip can be used to identify the presence of particular genes in a biological sample. These chips, containing hundreds or thousands of unique DNA probes are also called DNA microarrays and can be manufactured using a variety of techniques (e.g. semiconductor processing technology) on a variety of surfaces, including glass and plastic.

After hybridization, the probe array is washed, stained with streptavidin-conjugated phycoerythrin and scanned with the GeneArray® scanner. The expression profile is established with the presence and/or absence of the bound biotin*cDNA. Unfortunately, this approach is a solution-based system, in that tissue sample is homogenized. The prepared sample is then applied to the microarray. Unfortunately, the tissue sample is destroyed within this method and the expression gleaned from the array is that of a homogenized mixture of multiple cells rather than are analysis of cells as they are found in the body. While the above approached is somewhat helpful the destruction of the tissue sample's integrity prevents further analysis and the microarray detection of the mixture of cell material does not allow an analysis that is specific to certain cells.

A further approach is a bead based system developed by the Luminex Corporation as described in U.S. Pat. Nos. 6,268,222, 6,139,800, 6,057,107, 6,046,807, 5,981,180 and 5,736,330. This technology is the basis for the LabMAP™ (Laboratory Multi-Analyte Profiling technology). Luminex has developed internally dyed polystyrene microspheres with two spectrally distinct fluorochromes and using precise ratios, and has created 100 different microspheres. It is now possible to label each bead with a specific ligand and simultaneously detect up to 100 different analytes (e.g. protein, DNA, RNA, etc) in each sample. The detection and analysis is done in their microfluidics instrument, the Luminex¹⁰⁰ Analyzer.

This approach is also a solution-based system, in that the tissue sample is ground and then homogenized. The prepared sample is applied to the microspheres. Unfortunately, the tissue sample is destroyed within this method and the expression gleaned from the array is that of a homogenized mixture of multiple cells rather than an analysis of cells as they are found in the body While this approach is somewhat helpful, the destruction of the tissue sample prevents further analysis. Unfortunately, detection by the microspheres of the mixture of cell material does not permit cell-specific analysis.

An additional method of detection is a nanobarcode based system by Nanoplex Technologies, Inc. of Mountain View, Calif. Nanoplex has designed Nanobarcodes™ (NBCS) that are cylindrical metal nanoparticles with different stripes, widths and shapes as described in U.S. Pat. Nos. 6,242,264, 6,025,202 and 5,609,907. NBCS can be functionalized with proteins, nucleic acids, etc and identified using optical microscopy. Nanoplex claims they can label 10,000 nanobarcodes with different oligonucleotides and detect rapidly target nucleic acids in solution.

This approach is yet another solution-based system, in that the tissue sample is ground and then homogenized. The prepared sample is applied to NBCS. Unfortunately, the tissue sample is also destroyed with this method and the expression gleaned from the NBCS is that of a homogenized mixture of multiple cells rather than an analysis of cells as they are found in the body. While the above approach is somewhat helpful, the destruction of the tissue sample prevents further analysis that is cell-specific.

Traditional enzyme detection methods involve mixing a DNA sample with a specific enzyme and a DNA fragment of known sequence called a probe. There is one probe specific for each SNP to be identified and a signal generated during this reaction indicates the presence of a particular SNP. Researchers can perform these measurements in parallel using current multi-well plates. Multi-well plates are rectangular plastic plates which typically contain 96, 384 or 1536 micro-wells. Each micro-well functions as a test tube. An advantage of this approach is that researchers can analyze different DNA samples in parallel on the same multi-well plate. Unfortunately, it is usually not possible to measure more than one SNP in an individual well. Thus, the throughput of traditional enzyme methods is relatively low.

### SUMMARY OF INVENTION

Accordingly, it is an object of the invention to provide a novel method which comprises a process having one probe and one target detection. The method according to the invention applies multiple probes (i.e. tens, hundreds, or thousands) to each tissue sample. The main component of the inventive method is the utilization of a specific reagent system, which is described below as a “Probe-Tagging” system. The inventive method utilizes cleavable and non-cleavable linkers and specific tagging molecules that upon cleavage or dissociation are analyzed using automated detection systems.

It is an object of the invention that the Probe-Tag system can be detected with the help of specific tag molecules that have been cleaved or dissociated from target binding sites. According to the inventive method, peptide-based tag molecules may be detected with the utilization of a microplate or a slide-microarray coated with anti-peptide specific antibodies in a sandwich immunoassay format.

It is a further object of the invention that all components of the inventive method such as binding, binding and staining, evaluation, and report generation are performed by automated instruments. The results are generated in usable report formats that are compliant with good laboratory practices (GLP).

It is a still further object of the invention that methods of analysis are devoid of subjective and investigator specific conclusions. According to the invention, utilization of an automated binding instrument enables evaluation according to standard criteria. The utilization of automated instrumentation reduces investigator-to-investigator variation.

It is yet a further object of the invention that slides are analyzed with multiple probes. In human tissue screening test illustrated below, housekeeping gene products such as actin may be included to generate semi-quantitative results based on target analyte/actin ratios. This type of normalization can yield reproducible, semi-quantitative data.

According to the invention, probes are labeled with one or more tags. Both probes and tags may be any form of a molecule. They can be derived from both biological and non-biological sources. These molecules can be in the form of antibodies, proteins, peptides, DNA, RNA, Peptide Nucleic Acids (PNA) and organic and inorganic molecules. Each “Probe-Tag” may have the following structures:

One Link & Tag System

| (a) | (b) − Mol x |
|---|---|
| [Probe]− | {[Linker₁] − [Tag₁]} |

Double Link & Tag System

| (a) | (b₁) − Mol₁ | (b₂) − [Mol₂] |
|---|---|---|
| [Probe]− | {[Linker₁] − [Tag₁]}-- | {[Linker ₂] − [Tag₂]} |

Multiple Link & Tag System

| (a) | (b) − Mol x | (bₙ) − [Mol n] |
|---|---|---|
| [Probe]− | {[Linker₁] − [Tag₁]}-- | {[Linkerₙ] − [Tagₙ]} |

Probe Anti-Probe Tag System

| (a) | (ap) | (b) − Mol x |
|---|---|---|
| [Probe]− | [Anti-Probe] | {[Linker₁] − [Tag₁]} |

The (a) section contains the probe molecule. Probes may be in the form of antibody, protein, peptide, nucleic acid, DNA, RNA, PNA or any other organic or inorganic molecule. The probe is linked to the (b) section via a linker structure. The linking structure may be a covalent or non-covalent chemical bond. The (b) section is the part that has the body of a specific molecule. There may be one or more (b) sections. Each (b) section may be composed of a specific linking sequence and a “specific Tag-Structure. In an alternative embodiment the (a) section and (b) section are linked together using an anti-probe (ap)

In a further alternative embodiment, the cleavable probe-tag design is used in proximity based assay system. The proximity based assay system has a general structure as follows:

Probe-Tag Design for Proximity Based Assay System

[P¹→L←X^{bt}]

X: Specific tag for probe 1

L: Linker

P¹: Probe 1

X^{bt: Biotin} conjugated X Tag molecule

[Y→L←P²]

Y: Specific tag for probe 2

L: Linker

P²: Probe 2

A connecting probe (D) is used to link [P¹→L←X^{bt}] and [Y→L←P²] via their X and Y tags. The connecting probe (D) may be a complementary nucleic acid fragment that can hybridize and connect X and Y nucleic acid (oligonucleotides) fragments, such as below:

X:D:Y

P and L may be any molecule or structure such as antibodies, peptides, proteins, nucleic acids, or any other inorganic or organic molecule. Linker molecule may be hydrolyzed or dissociated via certain controlled conditions such as enzymes, reducing agents, etc. X, Y and D may be any form of a nucleic acid (i.e. DNA, RNA, PNA, etc) and they may be labeled with other molecules such as biotin or fluorescein isothiocyate (FITC). X and Y may also be a protein, peptide or any type of a molecule.

D may be an aptamer or an antibody with specific affinity against X and Y. D may also have multiple sections with different molecules such as DNA¹-Polymer-DNA². The polymer section gives the connecting probe the flexibility of distance control. The polymer section may be composed of different polymers such as nucleic acids, peptides, carbohydrates, poly(amino acids) or structures such as nanobarcodes and nanocrystals. The D section may also be a double stranded DNA fragment with specific oligonucleotide flanking regions at both ends. The function of connecting probe is to bind or hybridize simultaneously to specific tag molecules.

Proximity based assay system, according to invention, allows localizations of the respective probe molecules to cell types and other structures present in tissue samples. The connecting probe (D) connects the P¹ & P² probes when they are in “proximity” . Proximity means an appropriate distance for the connecting probe to connect the specific tag molecules X and Y. The presence and amount of connected P^{l} and P² probes can be used to localize one probe such as P¹ to the other probe P² and calculate the percent binding distribution of (P¹) in tissue sections. In this proximity based assay system, identifications, quantifications and determinations are achieved without the aid of a microscope. In this further alternative embodiment using a cleavable probe-tag design system, X and Y tags connected by the connecting probe are released from the tissue section by cleaving their respective linkers. The presence and amounts of X and Y can be quantitated.

The tags within all embodiments can be any form of a molecule such as peptides, proteins, DNA, RNA and other organic and inorganic structures. It is contemplated within the scope of the invention that structures such as beads, particles, vesicles, liposomes, oligonucleotides and nanobeads, nanobarcodes and nanocrystals (quantum dots) may also be utilized as tags.

According to the invention, the linking structure within all embodiments can be any chemical bond that is amenable to further manipulation. The linkers may be disulfide bonds (SPDP, SMPT), Protease specific peptide sequences, enzyme specific DNA/RNA sequences, pH sensitive structures/sequences or any other chemical or physical bond that is easily manipulated by dissociation, cleavage or hydrolyzation. In one illustrative embodiment the cleavable-linking structure is one (SPDP) that is available from Pierce Chemical (Rockford, Ill.). These cleavable linking structures include but are not limited to periodate-cleavable glycols (DST), dithionite-cleavable diazo bonds, hydroxylamine- cleavable esters (EGS) and based-labile sulfones (Hermanson, G., Bioconjugate Techniques, 1995, Page 292-296)

According to the invention, the inventive method enables one to screen multiple probes simultaneously on the same tissue section. The inventive method further allows this multiple probe screening without destruction of cells comprising the tissue sections.

In an alternative illustrative embodiment the probe molecules are labeled with one or more “tags” The probes and tags may be any form of a molecule such as antibodies, proteins, peptides, organic and inorganic molecules, DNA, RNA, PNA and other similar compounds. The linking structure/sequence may be any form that is cleavable or non-cleavable.

### DETAILED DESCRIPTION OF EXEMPLARY EMBODIMENTS

According to the present invention, the exemplary embodiments and examples of use and operations disclosed are discussed in terms of monoclonal antibodies, and more particularly, in terms of monoclonal antibodies having cleavable probes that are useful in the detection of various physiological disorders. It is contemplated within the scope of the invention that polyclonal antibodies can also be utilized. The present disclosure finds applications in a wide variety of assays used in the medical field including but not limited to those employed in the detection of melanoma, pancreas and breast cancers and will be better understood with reference to the following definitions:

“Tags”: shall mean any form of organic and inorganic molecules such as peptides, proteins, DNA/RNA, beads, particles, vesicles, liposomes and nanobeads and nanobarcodes;

“Linkers”: shall mean any form of linking structure or sequence that is amenable to further manipulation. The linkers may be disulfide bonds (SPDP, SMPT), protease specific peptide sequences, enzyme specific DNA/RNA sequences, pH sensitive structures/sequences or any other chemical or physical bond that is easily manipulated (dissociated, cleaved and/or hydrolyzed). Linkers may also be any form of linking structure or sequence that is not cleavable;

“Probes” shall mean any form of a molecule, biological and non-biological in nature such as antibodies, proteins, peptides, organic & inorganic molecules, DNA, RNA, PNA and all similar compounds;

“Target” shall be any type of organic or inorganic molecule such as, but not limited to, receptors, enzymes, antibodies, proteins, peptides, nucleic acids, lipids, small organic molecules, phage particles, viruses, microorganisms;

“Elution buffers” shall mean buffers that may be composed of decoupling agents such as reducing agent dithiothreitol (DTT) or dissociating agents such as low pH, high pH, high salt, urea, guanidine. HCI, potassium thiocyanite; and

“Anti-Probe” shall mean any form of a molecule that has high affinity against specific probes (primary molecules) such as but not limited to Goat anti-mouse for mouse IgG primary molecules; Mouse anti-myc for recombinant expressed proteins with c-myc epitope; Mouse anti HisG for recombinant protein with His-Tag epitope, Mouse anti Xpress for recombinant protein with epitope-tag, Rabbit anti goat for goat IgG primary molecules, Mouse anti Thio for Thioredoxin fusion proteins, Rabbit anti GFP for Fusion Protein, Jacalin for α-D-Galactose, and Melibiose for carbohydrate-binding proteins, Sugars, Nickel Couple Matrix and Heparin.

In an illustrative embodiment, a method of making a monoclonal antibody having a cleavable probe useful in detecting the presence of Lewis A (Type 1 chain) is disclosed. This monoclonal antibody detects the presence of the above blood group antigen (Lewis A), which is expressed by certain epithelial cells.

A method of generating the above cleavable antibody probe is accomplished using a heterobifunctional cross-linker. Heterobifunctional conjugation reagents are used to cross-link proteins and other molecules in a two or three-step process that limits the degree of polymerization often obtained using homobifunctional cross-linkers. A common conjugation scheme utilizing the above heterobifunctional crosslinker involves a protein being modified with the reagent using the cross-linker's amine-reactive end. The modified protein is purified removing excess reagent by rapid dialysis. After the purification step the sulfhydryl-reactive end of the cross-linker can be used to conjugate to a sulfhydryl-containing molecule.

Generation of a cleavable antibody-probe is accomplished by the following process. The probe, which in this illustrative embodiment is an antibody, is formed in the following manner. A mouse antibody is incubated with N-Succinimidyl 3-(2-pyridyldithio)propionate (SPDP) (Pierce Biotechnology, Rockford, Ill.) reagent in phosphate buffered saline (PBS) buffer for approximately 30-60 minutes at room temperature. The un-conjugated SPDP is removed via dialysis. The conjugated SPDP molecule is reduced with DTT to generate a —SH group thereby forming an antibody with a free —SH group.

Generation of a tag is accomplished by the following method. The tag molecule, which is this illustrated embodiment is a peptide, is incubated with a SPDP reagent in PBS buffer for approximately 3-60 minutes at room temperature. The un-conjugated SPDP is removed via dialysis thereby forming a tag molecule with a reactive SPDP group.

Generation of an antibody having a cleavable tag is accomplished by incubating the antibody with the free —SH group and the tag molecule with a reactive SPDP group in PBS for approximately 18-24 hours at room temperature. When needed, un-conjugated tag molecules are removed via dialysis or column chromatography forming an antibody with a cleavable tag. It is contemplated within the scope of this invention that generation of an antibody with a cleavable tag can be accomplished by applying the free —SH group to the tag molecule and the reactive SPDP group to the antibody thereby forming an antibody with a cleavable tag.

A tissue sample of interest is prepared according to generally acceptable protocols known within the art. Within this illustrative embodiment, the tissue sections are prepared as follows: the tissue sample is subjected to de-parrafinization, rehydration, peroxide blocking (as needed), and heat induced epitope retrieval (HIER) (as needed). Nonspecific binding sites are blocked with a specific block reagent such as BB+(2% BSA, 0.5% Tween 20, 0.05% Proclin 300, 0.75 mg/ml Casein, 1 mg/ml horse IgG, 1 mg/ml Sucrose diluted in PBS) and incubated approximately 2-20 minutes at room temperature or overnight at 4° C. The antibody-probe mixture is prepared as set forth above. Each antibody is prepared in the same block buffer and applied to the tissue sample at its optimum final binding concentration. The antibody probe and tissue sample are incubated at room temperature for approximately 10-60 min or overnight at 4° C. Unbound antibody-probes are washed with PBS or other buffers known in the art such as PBS with Tween 20, or Tris Buffer, etc.

The specifically bound antibody-Tags are eluted. Cleavable linkers are cleaved by adding a reducing agent such as DTT or TCEP (Pierce Biotechnolgy). DTT at 50 mM or less may be used to cleave all disulfide bonds. The reducing agent may be added a number of times to increase the elution yield.

The presence or absence of the tag molecule can be determined in a sandwich immunoassay, an enzyme-linked immunosorbent assay (ELISA), a dot blot assay, or any other commonly known immunoassay known in the art. The eluates are prepared as follows for ELISA detection: eluates with a low pH need to be neutralized and supplemented by carrier proteins such as the addition of BSA block (2% in PBS with Tween 20). Eluates with high DTT may need to be desalted or diluted. Alternatively, low DTT concentrations may be used for elution that does not require further manipulation. Both types of eluates may also need the addition of carrier proteins such as BSA (1-6 mg/ml).

The tissue sample eluates are applied to designated wells. The wells are coated with specific antibodies such as rabbit anti-Tag-1, Rabbit anti Tag-2, etc. There is a specific strip of wells for each probe. For example, one well strip (8 wells) will be coated with pure rabbit anti-Tag-1 Ab. According to the invention, Tag-1 standards are applied to the first six wells and tissue sample is applied to the remaining two wells. The wells are incubated at room temperature for approximately one hour or approximately eighteen hours at 4° C. The wells are washed approximately three to five times with wash buffer (PBS, 0.05% Tween 20). A conjugate reagent alone or in mixture is applied to the wells. In this illustrated embodiment a biotinylated form of a specific antibody, such as Bio-rabbit anti-Tag-1, bio-rabbit anti-Tag-2, and so forth are applied as the conjugate.

The wells are incubated at room temperature for approximately one to four hours and washed with wash buffer three to five times. A label reagent is applied (Streptavidin-HRP) and the well is incubated at room temperature for one to four hours. The well is washed with wash buffer three to five times. A HRP substrate mixture, o-Nitrophenyl-β-D-Galactopyranoside (OPD) is applied and incubated 10-20 minutes. After incubation, stop reagent (4.5M H₂SO₄) is applied and detection is accomplished using an ELISA Plate Reader. The results are quantified according to a standard curve for each peptide-probe.

An alternative illustrative embodiment utilizes double-tagged probes for sequential binding and then staining. Each probe has a primary and secondary tag containing distinct linkers. In this alternative illustrative embodiment there are three probes that are distinct antibodies. It is contemplated within the scope of the invention that the probes can be any organic or inorganic compound having the ability to form an antigenic reaction with a target. In this illustrative embodiment each probe has primary and secondary tags that are oligonucleotides. These oligonucleotides are distinct from each other.

In this embodiment the three probe-tag-tag compounds are applied to a tissue section and incubated. Unbound probes are washed with PBS. An elution buffer is applied to bound probes causing their secondary tag miolecule to be eluted. The secondary tags are connected via disulfide links that are eluted using a reducing agent such as DTT. The eluted secondary tag molecules are collected and identified using detection methods known in the art. The identification of secondary tag molecules determines the bound probe or probes' identity.

The probe of interest is later identified by applying a specific anti-tag molecule such as Biotin conjugated anti-Tag (complementary DNA) to the primary tag molecule. The specific anti-tag molecule is incubated and washed eliminating non-specific binding (NSB). A label reagent Streptavidin-HRP is applied and incubated and unbound label is washed. A substrate such as AEC (red precipitation) or DAB (brown precipitation) is applied, incubated. The tissue sample is washed counter stained and mounted for image analysis.

In a further illustrative embodiment a complex having a probe anti-probe having a cleavable detectable tag is formed in the following manner. Peptides used as tags are incubated separately with SPDP reagent in PBS buffer for approximately 30 minutes at room temperature. The un-conjugated SPDP is removed via desalting using a Sephadex G25 column. The SPDP molecule is reduced with DTT generating an active disulfhydryl group on the tag molecule. Any excess DTT is removed via desalting with a Spehadex G25 column. An anti-probe, which in this illustrative embodiment is Goat anti-mouse antibody (GAM), is incubated with SPDP reagent in PBS buffer for approximately 30 minutes at room temperature. Un-conjugated SPDP is removed via desalting on a Sephadex G25 column thereby forming an anti-probe with a reactive SPDP group.

The respective peptide tag molecule with the free sulfhydryl group and the anti-probe having a reactive SPDP group are incubated separately in PBS at room temperature for approximately 18 hours. This incubation results in the formation of multiple and separate Goat anti-mouse antibody (anti-probe) with unique-peptide tags. Un-conjugated tags may be removed via dialysis or chromatography methods known in the art.

Each “goat anti-mouse antibody with a unique-peptide tag” is then incubated with its respective probe for approximately 20 to 60 minutes at room temperature or overnight at 4° C. The probes in this illustrative embodiment are antibodies. Within this illustrative embodiment there are three separate antibody probes. It is contemplated within the scope of the invention that numerous distinct probes can be used having anti-probe complexes each having a unique tag. The antibody-anti-probe- tag complexes are prepared in the same block buffer and applied to the tissue sample at their optimum final binding concentration. During this dilution step, free unbound anti-probe sites are blocked with the addition of excess non-specific probe molecules such as non-immune mouse IgG for anti-probe GAM. After incubation, the pre-formed antibody-anti-probe complexes having unique tag molecules are mixed together and applied to a tissue sample of interest. Tissue sample and antibodies with anti-probes having tag molecules are incubated for approximately. 10 to 60 minutes at room temperature. The sample is washed and unbound antibodies with anti-probe having unique tags are discarded.

The sample bound antibody-tag molecules (probe:anti probe-tag complex) are eluted by the use of strong dissociating agents such as low pH buffers. In this illustrative embodiment a Glycine buffer having a pH of 2.7 and a concentration of 0.1M is used. It is contemplated within the scope of this invention that other buffers known in the art may be used. Dissociating buffers may also be used with the cleavable linker containing tags to elute the whole antibody-complex or “anti-probe-tag” structures instead of tag alone. After elution, low pH buffers may need to be neutralized with a neutralization buffer such as Tris Buffer (pH 8.0).

The presence or absence of a tag molecule can be determined using a sandwich immunoassay, ELISA, dot blot or any other commonly known immunoassay known in the art. The eluates are prepared as follows for ELISA detection: eluates with low pH need to be neutralized and supplemented by carrier proteins such as the addition of BSA block (2% in PBS with Tween 20) while eluates with denaturation agents (urea) may need to be desalted or diluted. Both types of eluates may need the addition of carrier proteins such as BSA (1-6 mg/ml). Tissue samples are applied to designated wells. The wells are coated with specific antibodies such as rabbit anti-Tag-1, Rabbit anti Tag-2, etc. There is a specific strip of wells for each probe. For example, one well strip (8 wells) will be coated with pure rabbit anti-Tag-1 Ab. According to the invention, “anti-probe-Tag-1” standards are serially diluted and applied to the first six wells and the tissue sample eluate is applied to the remaining two wells. The wells are incubated at room temperature for approximately one hour or approximately eighteen hours at 4° C. The wells are washed approximately three to five times with wash buffer (PBS, 0.05% Tween 20). A conjugate reagent mixture is applied to the washed wells.

In this illustrated embodiment where anti-probe tag molecules are being detected, a biotinylated form of a specific antibody, such as rabbit anti GAM antibody, is applied as a conjugate against the anti-probe. The wells are incubated at room temperature for approximately one to four hours and washed with wash buffer three to five times. A label reagent (Streptavidin-HRP) is applied and incubated at room temperature for one to four hours. The wells are washed with a buffer three to five times. A HRP substrate mixture OPD is applied and incubated 10-20 minutes. After incubation a stop reagent (4.5M H₂SO₄) is applied and detection is accomplished using an ELISA Plate Reader. The results are quantified according to a standard curve for each peptide-tag. In one illustrative example using the above probe-anti-probe-tag method the results are summarized below in table 1.

TABLE 1
Complex-Base Application
Components

| Tissues | Probes (Mouse anti-human) | Anti-Probes with Tags |
|---|---|---|
| Pancreas | Chromogranin (mChrom) | GAM-Lysozyme (GAM-Lys) |
| Melanoma | PCNA (mPCNA) | GAM-Carbonic Anhydrase |
| | | (GAM-CA) |

Assay Design

| Tissues | Complex | Unique Tag for Probe |
|---|---|---|
| Pancreas | {mChrom:GAM-Lys} | Lysozyme |
| | {mPCNA:GAM-CA} | Carbonic Anhydrase |
| Melanoma | {mChrom:GAM-Lys} | Lysozyme |
| | {mPCNA:GAM-CA} | Carbonic Anhydrase |

ELISA Results

| Tissues | Anti-Chromogranin Antibody | Anti-PCNA Antibody |
|---|---|---|
| Pancreas | +Binding | +/−Binding |
| Melanoma | −Binding | +Binding |

In an alternative non-quantitative detection method, eluted tag fractions are collected. Tissue sample eluate is applied to each dot blot well spot. According to the invention, a high affinity binding membrane such as nylon membranes (+Charge) is used. The membranes are blocked with a block buffer (PBS, 1% BSA, 0.05% Tween 20, 3% serum) and incubated for approximately one hour at room temperature. After incubation membranes are washed with a buffer having the composition of PBS, 0.05% Tween 20, 0.1% BSA. After washing a specific rabbit anti-Tag antibody (e.g. rabbit anti-Tag-1 diluted in block buffer) is applied to each well or strip. The well is incubated at room temperature for approximately one hour. The membranes are washed three to five times with a wash buffer.

The rabbit antibodies are detected with bio-goat anti rabbit antibody by applying the bio-goat anti rabbit antibodies and incubating the mixture at room temperature for approximately one hour. The membrane is washed again with a wash buffer three to five times. After washing a Strep-Alk Phos label is applied and incubated for approximately one hour at room temperature. The membrane is washed a third time for three to five times with wash buffer. After washing, an Alk-Phos enzyme substrate (NBT/BCIP) is applied and incubated for approximately 10-20 minutes at room temperature. After incubation the membrane is washed with water. The membrane is dried and scanned and stored for long term evaluation. Results are compared to known serially diluted tag concentrations.

Tissue sections (typical 4-5 micron) contain very little amount of material. Therefore it is contemplated within the scope of this invention that signal amplification systems will be used to increase signal-to-noise ratios. These amplification systems may include, but are not limited to, polymerase chain reaction (PCR) based amplifications. Immuno-PCR and real-time PCR are contemplated within the scope of this invention as ultra-sensitive quantitative methods to determine target molecules. Immuno-PCR is similar to conventional indirect ELISA except that the conjugate-antibody (Anti-probe such as goat anti-mouse) is conjugated to a short oligonucleotide (tag molecule) instead of biotin or an enzyme molecule. The oligonucleotide is captured and amplified via PCR.

Further amplification systems such as Polymer based amplifications: Anti-Probe and HRP coupled Dextrane polymer (Dako Corp., Carpinteria, Calif.) may be used to directly detect target probe concentrations. Poly-HRP-Streptavidin polymers (RDI, Flanders, N.J.) may be used to replace the label reagent (Streptavidin-HRP) increasing signal intensity.

Other amplification methods such as Rolling Circle Amplification (RCA) (Molecular Staging Inc., New Haven, Conn.) and Ramification Amplification Method (RAM) (Hamilton Thome Research, Inc. Beverly, Mass.) are contemplated within the scope of this invention. In the RCA procedure, an oligonucleotide primer (tag) is covalently attached to an antibody such as rabbit anti mouse antibody. This, oligo-tagged secondary antibody is used to detect bound mouse IgGs (primary antibodies) on target areas such as 96 well microarrays, ELISA, etc. Addition of circular DNA, DNA polymerase, and nucleotides initiates a linear-DNA extension and generates signal amplification. For example, a nucleotide mixture with FITC-12-dUTP will result in FITC-incorporated linear DNA fragments. Detection may be achieved with an enzyme-conjugated anti-FITC antibody.

It is contemplated within the scope of this invention that normalization of a tissue section will be based upon the tissue section's total area. Tissue sections, according to the inventive method, are counter-stained (hematoxyllin and eosin, to obtain color) and scanned. The scanned images are analyzed by an image analysis program (Scion Corp.). Each tissue section'sarea is used to normalize tissue-specific data.

Another method of normalization is the determination total of accessible protein content for each tissue section. The accessible portion is described as the tissue section accessible to antibody-probe reagents. To achieve this objective a modifying reagent is used such as FITC to label all accessible tissue proteins via their free amino groups. Fluorescamine concentrations are low enough as to not inhibit downstream antibody reactions. The amount of FITC conjugated proteins is determined with the use of “anti-FITC antibody-Linker-tag” similar to other antibody-tag reagents.

A further method of normalization utilizes house-keeping gene expression levels. In this type of normalization, common and well established house-keeping gene products are used such as beta Actin, Glyceraldehyde-3-phosphate Dehydrogenase (GAPDH), Beta-Tubulin, etc. For example; “Anti GAPDH Antibody-Probe” reagent may be used all tissue sections. As is the case above, individual antibody values will be normalized according to the each tissue section's GAPDH value.

Although the illustrative embodiments herein use the above described method of cleavable and non-cleavable attachment to bind antibodies with cleavable or non-cleavable tags, it should be appreciated that the method of using a heterobifunctional cross linker reagent described herein can be used to link other entities together in a cleavable/non-cleavable manner. This method of using a heterobifunctional cross linker can be applied to forming other cleavable entities such as antibody to antigen complexes, protein to protein complexes, protein to nucleic acid complexes, DNA to DNA complexes, DNA to RNA complexes, and substrate to enzymes complexes or other binding partner pairs. Similarly, other crosslinkers can be used to establish chemical bonds between entities that cari be manipulated to achieve a desired separation that is advantageous to the assay method.

The foregoing has been a description of certain specific embodiments of the present disclosure. The present disclosure is not to be limited in scope by the illustrative embodiments described which are intended as specific illustrations of individual aspects of the disclosure, and functionally equivalent methods and components are within the scope of the invention. Indeed, various modifications of the disclosure, in addition to those shown and described herein will become apparent to those skilled in the art from the foregoing description and all such modifications are included.

What is claimed is:

1. A method of forming a probe with a cleavable tag for use in an immunometric assay for determining the presence or concentration of a target in a sample comprising the steps of:
providing a compound capable of reacting with said target to be assayed;
modifying said compound with a linking agent forming a compound with linkage;
reducing said linkage with a reducing agent forming a compound with a reactive group;
providing a detectable tag;
modifying said tag with a linking agent forming a tag with a reactive group;
incubating said tag with a reactive group with said compound with said reactive group forming a compound having a cleavable tag specific for said target.

2. The method of claim 1 wherein said tag is a peptide.

3. The method of claim 1 wherein said compound is an antibody.

4. The method of claim 3 wherein said antibody is mouse anti-human BG-5 monoclonal specific antibody.

5. The method of claim 3 wherein said antibody is mouse anti-human AE1 monoclonal specific antibody.

6. The method of claim 1 where said linking agent is a heterobifunctional cross linking agent.

7. The method of claim 6 wherein said heterobifunctional cross linking agent is N-Succinimidyl 3-(2-pyridyldithio)propionate (SPDP).

8. The method of claim 1 wherein said reducing agent is Dithiothreitol.

9. The method of claim 1 wherein said tag is Lysozyme

10. The method of claim 1 wherein said tag is carbonic anhydrase.

11. The method of claim 1 wherein said tag is selected from the group consisting of peptides, proteins, carbohydrates, nucleic acids, DNA, RNA, PNA, oligonucleotides, beads, particles, vesicles, liposomes, nanobeads, nanobarcodes, nanocrystals.

12. The method of claim 1 wherein said compounds is selected from the group consisting of peptides, proteins, nucleic acid sequences, DNA, RNA, PNA carbohydrates, lipids, steroids.

13. The method of claim 1 wherein said compound having a cleavable tag specific for said target formed by the process is BG5-ss-Lysozyme.

14. The method of claim 1 wherein said a compound having a cleavable tag specific for said target formed by the process is AE1-ss-carbonic anhydrase.

15. A method for detecting antigens in tissue comprising the steps of:
providing an antibody specific for the antigen to be detected;
modifying said antibody wherein a cleavable tag is attached to said antibody forming an antibody with a cleavable tag;
incubating said tissue with said antibody with cleavable tag under conditions permitting an immunological reaction between said antibody with cleavable tag and said antigen to be detected thereby forming a complex;
cleaving said tag from said complex;
detecting said tag.

16. The method of claim 15 wherein said antibody is mouse anti-human BG-5 monoclonal specific antibody.

17. The method of claim 15 wherein said antibody is mouse anti-human AEI monoclonal specific antibody.

18. The method of claim 15 wherein said tag is Lysozyme

19. The method of claim 15 wherein said tag is carbonic anhydrase.

20. The method of claim 15 wherein said tag is selected from the group consisting of peptides, proteins, antigen complexes, nucleic acid complexes, DNA complexes, RNA complexes and enzyme complexes.

21. A method of forming a antibody with a cleavable tag for use in an immunometric assay for determining the presence or concentration of an antigenic substance in a sample comprising the steps of:
providing an antibody capable of immunologically reacting with said antigen to be assayed;
modifying said antibody with N-Succinimidyl 3-(2-pyridyldithio)propionate forming a reactive disulfide linkage;
reducing said disulfide linkage with Dithiothreitol forming an antibody with a free sulfhydryl group;
providing a detectable tag;
modifying said detectable tag with additional N-Succinimidyl 3-(2-puridyldithio)propionate to form a detectable tag with a reactive group;
incubating said detectable tag with a reactive group with said antibody with a free sulfhydryl group forming an antibody having a cleavable tag specific for said antigen.

22. The method of claim 21 wherein said tag is selected from a group consisting of lysozymne and carbonic anhydrase

23. The method of claim 21 wherein said antibody is mouse anti-human BG-5 monoclonal specific antibody.

24. The method of claim 21 wherein said antibody is mouse anti-human AE1 monoclonal specific antibody.

25. A method of forming an antibody with a cleavable tag for use in an immunometric assay for determining the presence or concentration of an antigenic substance in a sample which comprises:
providing antigen detection means capable of immunologically reacting with said antigen to be assayed;
modifying said antigen detection means forming a compound reactive linkage means;
reducing said reactive linkage means to form a free reactive group;
providing detectable tag means;
modifying said detectable tag means with reactive group means;
incubating said modified detectable tag means with said reactive group means with said free reactive group of said antigen detection means forming antigen detection means specific for said antigen to be assayed and having a cleavable tag.

26. The method of claim 25 wherein said detectable tag means is selected from the group consisting of Lysozyme and Carbonic Anhydrase.

27. The method of claim 25 wherein said antigen detection means is a mouse anti-human AE1 antibody.

28. The method of claim 25 wherein said antigen detection means is a mouse anti human BG5 antibody.

29. The method of claim 25 further including the step of concentrating said detectable tag prior to said detecting step.

30. A method for detecting antigens in tissue comprising the steps of:
providing antigen detection means, specific for the antigen to be detected and having cleavable marking means;
contacting said tissue with said antigen detection means under conditions permitting the formation of antigen-antigen detection means complex to be formed;
cleaving said marking means from said complex;
detecting said marking means to determine its presence or absence; and
correlating said presence or absence with the presence or absence of said antigen to be detected.

31. A method of forming a compound having a probe anti-probe complex having distinct cleavable tags for use in an immunometric assay for determining the presence of targets in a sample comprising the steps of:
providing an anti-probe capable of binding with multiple probes wherein said probes react with said target to be assayed;
modifying said anti-probe with a linking agent forming a complex with linkage;
reducing said linkage with a reducing agent forming an anti-probe with a reactive group;
providing at least one detectable tag;
modifying said detectable tag with a linking agent forming a detectable tag with a reactive group;
incubating said detectable tag with a reactive group with said anti-probe with said reactive group forming at least one anti-probe having a cleavable detectable tag;
providing at least one probe; and
incubating said probe with said anti-probe having a cleavable detectable tag forming a probe anti-probe complex having a cleavable detectable tag.

32. The method of claim 31 wherein said anti-probe is goat anti-mouse antibody.

33. The method of claim 31 wherein said detectable tag is selected from the group consisting of peptides, proteins, carbohydrates, nucleic acids, DNA, RNA, PNA, oligonucleotides, beads, particles, vesicles, liposomes, nanobeads, nanobarcodes, nanocrystals.

34. The method of claim 31 wherein said probe is an antibody.

35. The method of claim 31 wherein said complex is selected from the group consisting of Goat anti mouse IgG, Rabbit anti goat IgG, Mouse anti Xpress, Mouse anti HiG, Mouse anti myc, Mouse anti Thio and Rabbit anti GFP.

36. The method of claim 31 wherein said anti-probe is selected from the group consisting of Protein A and Protein G, Lectins, Sugars, Nickel coupled Matrix and Heparin

37. A method for detecting antigens in tissue comprising the steps of:
providing an anti-probe;
modifying said anti-probe wherein a cleavable detectable tag is attached to said anti-probe forming an anti-probe with a cleavable detectable tag;
incubating said anti-probe with a cleavable detectable tag with a probe forming a probe anti probe complex having a cleavable detectable tag;
incubating said tissue with said probe anti probe complex having a cleavable detectable tag under conditions permitting an immunological reaction between said probe anti probe complex having a cleavable detectable tag said antigen to be detected thereby forning a complex;
cleaving said tag from said complex;
detecting said tag.

38. A method of using a probe with a cleavable tag and a connecting probe for use in proximity based assay system for determining the localization of bound-probe on a target tissue comprising the steps of:
providing two or more probes having cleavably linked tag molecules wherein said probes are capable of reacting with said target to be assayed;
providing a connecting probe capable of connecting said tag molecules and providing conditions necessary for said connecting probe to connect said tag molecules;
cleaving said linked tag molecules and releasing said connecting probe with connected tags
capturing said connecting probe with connected tags; and
detecting and quantifying said connecting probe with said connected tags.

39. The method of claim 38 wherein said probe is an antibody, peptide, protein, nucleic acid, DNA, RNA, carbohydrates, organic molecule and in-organic molecule.

40. The method of claim 38 wherein said tag molecule is selected from the group consisting of oligonucleotide, nucleic acid, DNA, RNA, PNA, peptide, protein.

41. The method of claim 38 wherein said connecting probe is selected from the group consisting of oligonucleotide, nucleic acid, DNA, RNA, PNA, protein, aptamer, antibody;

42. The method of claim 38 wherein said connecting probe is a polymer.

43. The method of claim 42 wherein said polymer is selected from the group consisting of nucleic acid, DNA, RNA, PNA, protein, antibody; nanobarcodes, beads, nanocrystals.

44. The method of claim 38 wherein said connecting probe can connect specific tag molecules within proximity.
